(19) United States
(12) Patent Application Publication
(10) Pub. No.: US 2020/0377856 A1
(43) Pub. Date: Dec. 3, 2020

(54) Genome Edited iPSC-Derived Monocytes Expressing Trophic Factors

(71) Applicant: Guangbin Xia, Albuquerque, NM (US)

(72) Inventor: Guangbin Xia, Albuquerque, NM (US)

(73) Assignee: STC.UNM, Albuquerque, NM (US)

(21) Appl. No.: 15/733,108

(22) PCT Filed: Nov. 16, 2018
(86) PCT No.: PCT/US18/61481

§ 371 Date: May 18, 2020

Related U.S. Application Data
(60) Provisional application No. 62/587,530, filed on Nov. 17, 2017.

(51) Int. Cl. C12N 5/0786

(52) U.S. Cl. CPC C12N 5/0645; C12N 2510/00; C12N 2506/45

(57) ABSTRACT

Trophic factor expressing monocyte cells derived from gene-edited induced pluripotent stem cells, methods for making and using.

### CROSS-REFERENCE TO RELATED APPLICATIONS

The following application claims benefit of U.S. Provisional Application No. 62/587,514, filed Nov. 17, 2017, which is hereby incorporated by reference in its entirety.

### STATEMENT REGARDING GOVERNMENT SPONSORED RESEARCH

This invention was made with Government support under Grant No. K08 AR064836 awarded by NIH/NIAMS The U.S. Government has certain rights in this invention.

### BACKGROUND

The presence of monocyte/macrophages is indispensable for skeletal muscle regeneration¹⁻⁵ Mice deficient in chemokine receptor or ligand show impaired muscle regeneration, which is associated with a dramatic decrease in macrophage infiltration into the muscles and was reversed by wild type bone marrow transplantation^{5,6}. Depletion of circulating monocytes at the time of muscle injury totally prevents muscle regeneration^{4,6}. Patrolling monocytes selectively traffic to the sites of muscle degeneration/inflammation and convert into macrophages. Initially, these macrophages present as pro-inflammatory macrophage (M1) that will clear muscle debris and stimulate myogenic cell proliferation. Then, the phagocytosis of muscle debris induces a switch of pro-inflammatory M1 toward an anti-inflammatory phenotype (M2), which proliferate and promote muscle differentiation⁴. Macrophages also improve survival, proliferation and migration of engrafted myogenic precursor cells³. In individuals suffering from neurodegenerative disorders such as Myotonic dystrophy (Dystrophia Mytonica, DM) type 1 (DMI), there is infiltration of monocytes/macrophages⁷. Accordingly, the attraction of monocytes/macrophages to injured muscle provides an opportunity to introduce trophic factors through systemic administration of monocytes which have been bioengineered to deliver such factors directly to the areas that need them the most.

### SUMMARY

According to an embodiment the present disclosure provides monocyte cells which have been bioengineered to express trophic factors beneficial to muscle cell regeneration, transplantation, growth, and/or overall health, and methods for producing and using the same. According to some embodiments, the monocyte cells are derived from bioengineered induced pluripotent stem cells (iPSCs). According to further embodiments, the iPSCs may be derived from a cell sample obtained from a patient to be treated with the bioengineered monocyte cells.

### BRIEF DESCRIPTION OF THE DRAWINGS

FIG. 1 is an image of the results of genomic PCR performed on genome edited induced pluripotent stem cells (iPSC) expressing IGF-1 or Igk-IGF-1. RFP is a negative control with only red fluorescence cassette.

FIG. 2 is an image of the results of RT-PCR performed on genome edited iPSC-express IGF-1 or Igk-IGF-1. RFP is a negative control with only red fluorescence cassette.

FIG. 3 is an image of the results of Western Blot analysis on iPSC-derived embryoid bodies (EB) genome edited to express IGF-1 or Igk-IGF-1. RFP is a negative control with only red fluorescence cassette.

FIG. 4 is a schematic illustration of insertion cassettes used to insert IGF-1 or Igk-IGF-1 into iPSC cells.

FIG. 5 is an image of exemplary embryoid bodies and monocyte cells of the present disclosure.

### DETAILED DESCRIPTION

According to an embodiment the present disclosure provides monocyte cells which have been bioengineered to express factors beneficial to muscle cell regeneration, transplantation, growth, and/or overall health, and methods for producing and using the same. The disclosure further provides a method for improving muscle regeneration or transplantation by the introduction/delivery of bioengineered monocyte cells to an affected area.

As stated above, monocyte cells selectively traffic towards areas of muscle degeneration/inflammation and thus are naturally drawn towards areas where muscle regeneration/transplantation takes place. Accordingly, monocyte cells that have been engineered to produce factors associated with muscle cell muscle regeneration, growth, and/or overall health provide an excellent opportunity to provide a favorable environment for muscle regeneration for inherited muscular dystrophies, myopathies, and muscle injuries, aging-related sarcopenia, or other condition that causes muscle volume loss, injury, or other concerns. Of course it will be appreciated that while much of the disclosure is directed towards the treatment of muscle development and muscle disorders, the genetically engineered monocytes of the present disclosure could also be useful in treatment of amelioration of other disorders or other conditions/symptoms associated with those or other disorders. For example, the engineered monocytes of the present disclosure could be useful to help or encourage cellular regeneration in the central nervous system and/or other areas of the body.

One critical trophic factor for muscle regeneration/development is Insulin-like growth factor 1 (IGF-1), which has been implicated as central regulator of muscle regeneration. It is an important factor in in vitro skeletal muscle progenitor cells (SMPC) differentiation from iPSCs. IGF-1 accelerates muscle regeneration and restores muscle function and architecture by prolonging the regenerative potential of skeletal muscle through increasing satellite cell activity, recruiting circulating stem cells, modulating inflammatory factors, reducing muscle necrosis and fibrosis, and activating signaling pathways associated with muscle survival and regenerationn¹¹⁻¹⁸. The beneficial effects of local expression of IGF-1 on muscle regeneration was shown in degenerative processes such as muscular dystrophy, Amyotrophic Lateral Sclerosis, and sarcopenia related to aging.

Other factors that could be expressed by the bioengineered monocytes of the present disclosure include, but are not limited to, Fibroblast growth factor (FGF), platelet-derived growth factor (PDGF), transforming growth factor beta (TGF beta), Hepatocyte growth factor (HGF), nerve growth factor (NGF) and other neurotrophic factors, which play key roles in the muscle regeneration; and also brain-derived neurotrophic factor (BDNF) and Glia cell-derived neurotrophic factor (GDNF) and other neurotrophic factors, which are useful in the central nervous system such as neurodegenerative disorders (motor neuron disease, Parkinson disease, Alzheimer disease, spinocerebellar ataxia), neuroinflammatory diseases (Multiple sclerosis), and stroke. Accordingly, the present disclosure provides monocyte cells which have been genetically engineered to express factors such as, but not limited to, IGF-1, FGF, PDGF, TFG beta, HGF, NGF, BDNF and/or GDNF.

According to various embodiments, the bioengineered monocytes are derived from genetically altered cells capable of differentiating into monocyte cells. Examples of cells capable of differentiating into monocyte cells include, but are not necessarily limited to, induced pluripotent stem cells (iPSCs), embryonic stem cells, mesenchymal stem cells, or engineered somatic cells. See also, Kastenberg Z. J., et al, (2008) Alternative sources of pluripotency: science, ethics, and stem cells. Transplant Rev (Orlando) 22,215-222, which is hereby incorporated by reference for all purposes. Alternatively, the monocytes could be derived from hematopoietic stem cells or directly from peripheral blood. According to various embodiments, the cells from which the bioengineered monocytes may be derived from the individual who will be receiving the bioengineered monocytes, so as to minimize the likelihood or rejection or bio-incompatibility.

According to various embodiments, the monocytes or cells capable of differentiating into monocytes are genetically edited to express the desired factors. Numerous genome editing techniques have been developed and several are becoming increasingly well-known for their efficacy and utility in both in vitro and in vivo applications. Exemplary genome editing techniques typically rely on engineered nucleases such as meganucleases, zinc finger nucleases (ZFNs), transcription activator-like effector-base nucleases (TALENs) and the clustered regularly interspaced short palindromic repeats (CRISPR/Cas9) system to insert “donor” genetic material, typically in the form of an “insertion cassette” into a specific location of a “recipient” genome. Accordingly, these genome editing techniques can be used to insert a gene cassette encoding the desired trophic factor(s) into the genome of monocytes or cells that can be differentiated into monocyte cells. These genome editing techniques may incorporate viral (adenovirus, lentivirus) or non-viral methods (electroporation, lipid particles, or nanoparticles.)

According to a specific embodiment, the bioengineered monocytes are derived from iPSCs. iPSCs are similar to embryonic stem cells (ESC) in that iPSCs can be expanded indefinitely at the pluripotent stage and are able to differentiate into all three primary germ layers and, therefore, potentially into all the cell types of the body. The advantage of iPSC is the prospect of generating unlimited quantities of specific cell population for regenerative purposes. iPSCs are derived from somatic cells and the process does not involve the use of embryonic cells, removing ethnical concerns.

Moreover, iPSC cells can be derived from patient samples that are easily and even non-invasively obtained like skin, saliva, blood, or urine samples. Specific methods for generating iPSC cells are provided in Xia, G, et al. (2013). Generation of neural cells from DM1 induced pluripotent stem cells as cellular model for the study of central nervous system neuropathogenesis. Cell Reprogram 15: 166-177; and Zhou Y Y et al., Integration-free methods for generating induced pluripotent stem cells. Genomics Proteomics Bioinformatics. 2013 Oct; 11(5):284-7. doi: 10.1016/j.gpb.2013.09.008, each of which is hereby incorporated by reference for all purposes.

The iPSCs can be cultured using suitable culturing conditions. For example, iPSCs can be maintained using protocols such as those disclosed in Gao Y, Guo X, Santostefano K et al. Genome Therapy of Myotonic Dystrophy Type 1 iPS Cells for Development of Autologous Stem Cell Therapy. Mol Ther. 2016; 24:1378-1387; Xia G, Gao Y, Jin S et al. Genome modification leads to phenotype reversal in human myotonic dystrophy type 1 induced pluripotent stem cell-derived neural stem cells. Stem Cells. 2015; 33:1829-1838; Xia G, Santostefano K, Hamazaki T et al. Generation of human-induced pluripotent stem cells to model spinocerebellar ataxia type 2 in vitro. J Mol Neurosci. 2013; 51:237-248; and Xia G, Santostefano K E, Goodwin M et al. Generation of neural cells from DM1 induced pluripotent stem cells as cellular model for the study of central nervous system neuropathogenesis. Cell Reprogram. 2013; 15:166-177, each of which is incorporated by reference. According to some embodiments, these protocols may be modified to meet the criteria of clinically-clean iPSCs, including the use of feeder-free, xeno-free culture and coating media. While common cultures call for the use of an extracellular matrix such as, for example, the Corning Matrigel matrix (Corning, New York, N.Y.), it should be noted that the Corning Matrigel matrix contains a mixture of matrix proteins and growth factors of non-human origin. Accordingly, for applications wherein the cells are ultimately to be implanted in a human subject, it may be desirable to use cultures conditions that do not utilize non-human origin additives. According to a specific example, cultured cells may be coated with laminin and collagen IV from human cell culture (for example, Sigma-Aldrich C6745, Sigma-Aldrich Co.) and adapted to Laminin 521 coating culture conditions. Laminin 521 (LaminStem™ 521,05-753-1F, Biological Industries) is a chemically defined, animal component-free, xeno-free matrix. Those of skill in the art will be familiar with other suitable culturing conditions as well as the adaptation of those conditions for the specific uses of the presently described genome corrected cells.

In a specific example, the iPSCs are altered by targeted insertion of an IGF-1 gene cassette using a cytomegalovirus (CMV) promoter or other potent promoters in the safe harbor locus (for example the AAVS1 locus or the chemokine (C-C motif) receptor 5 (CCR5 gene) of the genome mediated by a site-specific gRNA-CRISPR/Cas9 system.

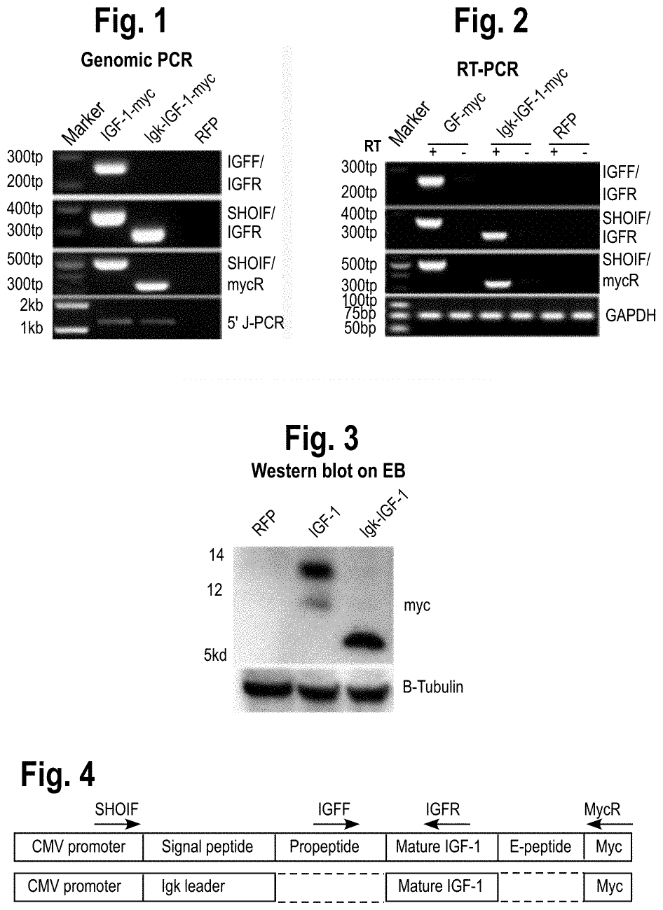

FIGS. 1-2 are Junctional PCR, RT-PCR respectively, showing correct insertion and expression of IFG-1 in human iPSCs genome edited to include a full length IFG-1 and Igκ-IGF-1 cassette inserted in the AAVS1 site. FIG. 3 shows the expression of IGF-1 protein in the genome-edited human iPSCs. FIG. 4 is a schematic view of the cassettes and position of the PCR primers that were used. In the depicted embodiment, both a full protein (IGF-1) and a secretary form of IGF-1 (Igκ-IGF-1) are constructed. E-peptides control IGF-1 bioavailability by preventing systemic circulation, offering a potentially powerful way to tether IGF-1 and other therapeutic proteins to the site of synthesis. In the depicted embodiment, c-myc is tagged to verify the expression of IGF-1. The c-myc tag also helps to identify and quantify local infiltrated monocytes after systemic injection.

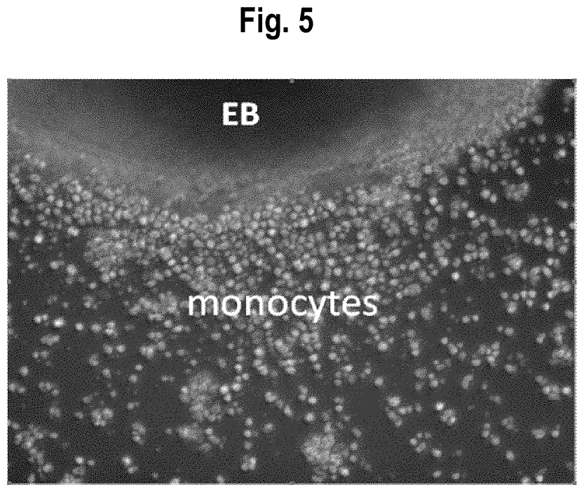

The genetically altered iPSC colonies can then be cultured for harvest as needed to obtain the genetically altered monocyte cells. As a specific example, iPSC colonies are detached and resuspended in embryoid body (EB) culture medium containing BMP-4 (50 ng/ml), VEGF (50ng/ml), FGF (10 ng/ml) and Y-27632 (10 μM) at a concentration of 1.2×105. 100 μl is then seeded to into 96-well ultra-low adherence plate for EB formation. After four days of EB differentiation, EBs are transferred into six-well tissue-culture plate (8 EBs per well) and cultured in differentiation medium (containing IL-3 (25-50 ng/ml) and M-CSF (50-100 ng/ml)). After four days, 4 ml of the differentiation medium will be added, and monocyte can be harvested at day 8. The medium will be replaced fresh and monocyte can then be harvested every 8 days as shown in the image in FIG. 5.

Differentiation of the genetically altered iPSCs into monocyte cells can also be achieved using methods described in, for example: Lachmann N., et al., Large-scale hematopoietic differentiation of human induced pluripotent stem cells provides granulocytes or macrophages for cell replacement therapies. Stem Cell Reports. 2015; 4:282-296; Yanagimachi M D., et al. Robust and highly-efficient differentiation of functional monocytic cells from human pluripotent stem cells under serum- and feeder cell-free conditions. PLoS One. 2013; 8:e59243; van Wilgenburg B., et al., Efficient, long term production of monocyte-derived macrophages from human pluripotent stem cells under partly-defined and fully-defined conditions. PLoS One. 2013; 8:e71098; Karlsson K R., et al. Homogeneous monocytes and macrophages from human embryonic stem cells following coculture-free differentiation in M-CSF and IL-3. Exp Hematol. 2008; 36:1167-1175; and Buchrieser J, et al., Human Induced Pluripotent Stem Cell-Derived Macrophages Share Ontogeny with MYB-Independent Tissue-Resident Macrophages. Stem Cell Reports. 2017; 8:334-345, each of which is hereby incorporated by reference for all purposes.

The IGF-1 monocytes can then be injected into the patient, for example, via system intravenous (IV) delivery, to improve muscle regeneration, transplantation, growth, etc. Importantly, because the monocyte cells are programmed to travel to areas of muscle injury, inflammation, etc.

Human iPSC-derived monocytes/macrophages resemble anti-inflammatory M2-polarized macrophages expressing classical macrophage markers (CD45, CD 14, and CD 163,)¹³⁸. These cells share ontogeny with MYB-independent tissue-resident macrophages¹⁴², which will stay longer in the tissue than bone marrow hematopoietic stem cell-derived monocytes/macrophages. Accordingly, the iPSC-derived IGF-1 producing monocytes/macrophages should exert long term effects. Moreover, the simplicity of the above-described technique enables the production of IFG-1 producing monocytes from iPSCs in large quantities, making them a viable treatment option for a variety of conditions and diseases.

Of course while the specific embodiment above described the production and use of IFG-1 producing monocyte cells, it will be understood that similar techniques could be used to produce engineered monocyte cells expressing any suitable factor including those identified above as being of interest and such engineered monocyte cells would be considered to be within the scope of the present disclosure.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

### REFERENCES

1. Summan M, Warren G L, Mercer R R et al. Macrophages and skeletal muscle regeneration: a clodronate-containing liposome depletion study. Am J Physiol Regul Integr Comp Physiol. 2006; 290:R1488-1495
2. Tidball J G, Wehling-Henricks M. Macrophages promote muscle membrane repair and muscle fibre growth and regeneration during modified muscle loading in mice in vivo. J Physiol. 2007; 578:327-336
3. Lesault P F, Theret M, Magnan M et al. Macrophages improve survival, proliferation and migration of engrafted myogenic precursor cells into MDX skeletal muscle. PLoS One. 2012; 7:e46698
4. Arnold L, Henry A, Poron F et al. Inflammatory monocytes recruited after skeletal muscle injury switch into antiinflammatory macrophages to support myogenesis. J Exp Med. 2007; 204:1057-1069
5. Martinez CO, McHale M J, Wells J T et al. Regulation of skeletal muscle regeneration by CCR2-activating chemokines is directly related to macrophage recruitment. Am J Physiol Regul Integr Comp Physiol. 2010; 299:R832-842
6. Zhao W, Lu H, Wang X et al. CX3CR1 deficiency delays acute skeletal muscle injury repair by impairing macrophage functions. FASEB J. 2016; 30:380-393
7. Finol H, Torres S, Rabucha A, Saenz H. Intramuscular capillary abnormalities in a case of myotonic dystrophy (Steinert's disease). Acta Cient Venez. 1992; 43:284-289

What is claimed is:

1. An induced pluripotent stem cell (iPSC)-derived monocyte cell that has been bioengineered to express one or more trophic factors.

2. The iPSC-derived monocyte cell of claim 1 wherein at least one of the one or more factors is associated with muscle growth or regeneration.

3. The iPSC-derived monocyte of claim 1 wherein the factors are selected from the group consisting of: Insulin-like growth factor 1 (IGF-1), Fibroblast growth factor (FGF), platelet-derived growth factor (PDGF), transforming growth factor beta (TGF beta), Hepatocyte growth factor (HGF), nerve growth factor (NGF), brain-derived neurotrophic factor (BDNF) and Glia cell-derived neurotrophic factor (GCNF).

4. The iPSC-derived monocyte of claim 1 wherein at least one of the factors is IFG-1.

5. The iPSC-derived monocyte of claim 1 wherein the cell was bioengineered via genome editing of the parent iPSC cell.

6. The iPSC-derived monocyte of claim 5, wherein genome editing comprises insertion of a cassette comprising the gene for the one or more trophic factors.

7. The iPSC-derived monocyte of claim 6 wherein the trophic factor is selected from the group consisting of: IGF-1, FGF, PDGF, TGF beta, HGF, NGF, BDNF and GCNF.

8. A method for treating or ameliorating a condition or disease comprising injecting a patient with the iPSC-derived monocyte cells of claim 1.

9. The method of claim 8 wherein a symptom of the disease or condition is muscle loss or degeneration.

10. The method of claim 9 wherein the trophic factor is associated with muscle growth or regeneration.

11. The method of claim 8 wherein the bioengineered monocyte cells are obtained by:
obtaining a cell sample from a patient with a condition or disease;
producing induced pluripotent stem cells (iPSCs) from the cell sample;
inserting a gene into the genome of the iPSCs to produce genetically altered iPSCs, wherein the gene being inserted will, in monocyte cells derived from the iPSCs, cause the monocyte cells to express one or more trophic factors that treat or ameliorate the condition or disease; and
differentiating the iPSCs into bioengineered monocyte cells.

12. The method of claim 11 wherein the one or more factors are selected from the group consisting of: IGF-1, FGF, PDGF, TGF beta, HGF, NGF, BDNF and GCNF.

13. The method of claim 11 wherein at least one of the factors is IFG-1.

14. A method for producing bioengineered monocytes comprising:
providing iPSC cells;
inserting a gene into the genome of the iPSCs to produce genetically altered iPSCs, wherein the gene being inserted will, in monocyte cells derived from the iPSCs, cause the monocyte cells to express one or more trophic factors; and
differentiating the iPSCs into bioengineered monocyte cells.

15. The method of claim 14 wherein the trophic factors are selected from the group consisting of: IGF-1, FGF, PDGF, TGF beta, HGF, NGF, BDNF and GCNF.

16. The method of claim 14 wherein the iPSC cells are obtained from a patient into whom the bioengineered monocyte cells are to be injected.
